(19) United States
(12) Patent Application Publication
(10) Pub. No.: US 2012/0244555 A1
(43) Pub. Date: Sep. 27, 2012

(54) METHOD OF DIAGNOSING MILD TRAUMATIC BRAIN INJURY

(75) Inventors: Brian J. Blyth, Fairport, NY (US); Jeffrey J. Bazarian, Honeoye Falls, NY (US)

(73) Assignee: University of Rochester, Rochester, NY (US)

(21) Appl. No.: 13/430,365

(22) Filed: Mar. 26, 2012

Related U.S. Application Data
(60) Provisional application No. 61/467,224, filed on Mar. 24, 2011.

(51) Int. Cl. G01N 33/92; G01N 27/447; G01N 21/76

(52) U.S. Cl. 435/7.9; 436/501; 204/606

(57) ABSTRACT

The present invention relates to a method of determining whether a subject has suffered a mild traumatic brain injury. The method comprises selecting a subject exposed to a head trauma; and determining whether a body fluid sample obtained from the selected subject comprises smaller than normal high density lipoprotein (HDL) particles, larger than normal HDL particles, or both; wherein detection of the smaller than normal HDL particles, larger than normal HDL particles, or both, indicates that the subject has suffered a mild traumatic brain injury.

This application claims the benefit of U.S. Provisional Patent Application Ser. No. 61/467,224 filed Mar. 24, 2011, which is hereby incorporated by reference in its entirety.

This invention was made with government support under National Institutes of Health U.S. Public Health Service Grants K23 NS41952 and R01 HD051865. The government has certain rights in the invention.

### FIELD OF THE INVENTION

This invention relates to a diagnostic method of determining whether a subject has suffered mild traumatic brain injury by measuring high density lipoprotein (“HDL”) particle size.

### BACKGROUND OF THE INVENTION

Mild traumatic brain injury (mTBI) affects 1.7 million patients annually in the United States (Faul et al., “Traumatic Brain Injury in the United States: Emergency Department Visits, Hospitalizations and Deaths 2002-2006,” Centers for Disease Control and Prevention, National Center for Injury Prevention and Control, Atlanta, Ga. (2010)) and is a significant risk factor for the development of neurodegenerative illness (Nemetz et al., “Traumatic Brain Injury and Time to Onset of Alzheimer's Disease: A Population-Based Study,” Am. J. Epidemiol. 149:32-40 (1999)). Diagnosis is made subjectively because there are no consistent objective findings associated with mTBI (THE CONCUSSION/MTBI WORKING GROUP, VA/DOD CLINICAL PRACTICE GUIDELINE FOR MANAGEMENT OF CONCUSSION/MILD TRAUMATIC BRAIN INJURY (2009)). Inaccurate diagnosis is common. Patients suffering mTBI due to combat or sports often deliberately under report their symptoms to avoid being separated from their team members. Because mTBI frequently results in subtle acute cognitive deficits, these patients are at risk for further injury. An intense search for accurate and clinically useful molecular biomarkers has largely failed, likely due to the presence of the blood brain barrier (BBB) which prevents passage of most molecules from brain into the peripheral circulation (Morganti-Kossmann et al., “TGF-beta is Elevated in the CSF of Patients with Severe Traumatic Brain Injuries and Parallels Blood-Brain Barrier Function,” J. Neurotrauma 16:617-628 (1999); Blyth et al., “Validation of Serum Markers for Blood-Brain Barrier Disruption in Traumatic Brain Injury,” J. Neurotrauma. 26:1497-1507 (2009)).

TBI has been called the “signature injury” of the current conflicts in Iraq and Afghanistan. Nearly 90% of these injuries are classified as mild or a concussion. Acutely and sub-acutely, mTBI often leads to subtle cognitive dysfunction. This post-mTBI cognitive dysfunction is particularly problematic in military populations where the injured subject is often making decisions for himself or a group that have life and death consequences. Diagnosis of mTBI is based on a clinical history alone. Reliable objective aids for the diagnosis of mTBI are not available. Military personnel in combat situations are often unwilling to provide an accurate history after mTBI because they do not want to abandon their units or, conversely, they wish to avoid further combat. Thus, there is a need for a reliable and objective test for the diagnosis of mTBI. Such a test would have substantial utility both in military populations in war zones as well as in civilians presenting to emergency departments. Additionally, an objective test would be useful in outpatient populations to identify the need for further hospital-based diagnosis and treatment.

The present invention overcomes these and other deficiencies in the art.

### SUMMARY OF THE INVENTION

One aspect of the present invention is directed to a method of determining whether a subject has suffered a mild traumatic brain injury. This method includes: selecting a subject exposed to a head trauma; and determining whether a body fluid sample obtained from the selected subject comprises smaller than normal high density lipoprotein (HDL) particles, larger than normal HDL particles, or both, where the detection of the smaller and/or larger than normal HDL particles indicates that the subject has suffered a mild traumatic brain injury.

In one embodiment, a determination is made concerning the presence of smaller than normal HDL particles in the body fluid sample.

In another embodiment, a determination is made concerning the presence of larger than normal HDL particles in the body fluid sample.

In a further embodiment, a determination is made concerning the presence of both the smaller than normal HDL particles and the larger than normal HDL particles in the body fluid sample.

Current diagnosis of mTBI requires an accurate clinical history. Confounding factors such as conditions mimicking TBI like syncope, intoxication, or seizures complicate clinical diagnosis of mTBI. Willful misreporting of symptoms is also a significant problem particularly with athletes and military personnel. For example, military personnel in Iraq and Afghanistan conflicts often rehearse answers to the standard field screening test for concussion in order to successfully answer in the event they are impaired following a concussion. The present invention circumvents these problems through application of an objective test.

The accompanying Examples demonstrate that unique populations of small HDL particles (less than about 7.2 nm via gel electrophoresis) and extremely large HDL particles (greater than about 12.8 nm via gel electrophoresis) represent useful biomarkers associated with, or indicative of, mild traumatic brain injury. These biomarkers for mTBI can be used for a highly selective and accurate diagnosis of mTBI, and obviate the influence of the BBB. Presence of these small HDL particles or large HDL particles, or both, in a body fluid sample from a head trauma patient is useful as an objective test for the diagnosis of mTBI. While S100B is a less accurate test for diagnosis of mTBI, it continues to be valuable to identify a subset of patients at high risk for traumatic injuries detectable with cranial computed tomography (CT). Therefore, combining detection of HDL particle size-based assessment of mTBI with S100B-based assessment can be useful for assessing mTBI patients generally and those at high risk for trauma detectable with cranial CT scans.

### BRIEF DESCRIPTION OF THE DRAWINGS

FIGS. 1A-B show a separation and analysis of serum containing lipoproteins fractionated by gel filtration. FIG. 1A illustrates results of pooled sera from uninjured (n=4) and mTBI (n=4) subjects that were analyzed by FPLC and the cholesterol content of each fraction was measured and plotted against fraction number. Based on size, major lipoprotein fractions are separated into VLDL, LDL, and HDL. FIG. 1B shows ApoA-1 content of fractions spanning the HDL peak that were analyzed by immunoblotting and quantified by densitometry.

FIG. 2 illustrates separation of serum lipoproteins by gradient gel electrophoresis under non-denaturing conditions from three uninjured control subjects and five mTBI subjects. After separation, proteins were electrophoretically transferred to PVDF membranes and apoA-1 was detected by immunoblotting. Sizes of apoA-1 containing particles ranged from 7 to 17 nm in diameter based on comparison with co-electrophoresed molecular mass standards as indicated.

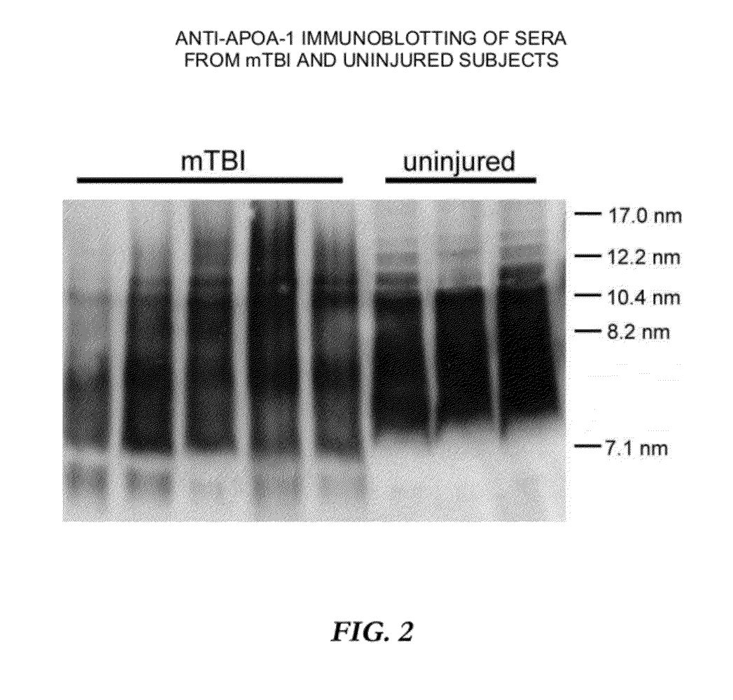

### DETAILED DESCRIPTION OF THE INVENTION

The present invention relates to methods of determining whether a subject has suffered a mild traumatic brain injury (mTBI). The methods include selecting a subject exposed to a head trauma, and then determining the presence of or level of a particular biomarker in a body fluid sample obtained from the selected subject. Presence of the biomarker or elevated levels of the biomarker in the body fluid sample indicates that the subject has suffered mTBI.

A subject exposed to a head trauma includes any mammal, preferably human, that is conscious or unconscious but not comatose. The subject who is exposed to the head trauma may exhibit extra-cranial injuries or may exhibit no extra-cranial injuries.

The method of the present invention can be practiced on patients whose head trauma is produced, at least in part, by brain injuries including those produced by blunt head trauma or missile penetration.

Conscious, as used herein, has the conventional meaning, as set forth in Plum, et al., The Diagnosis of Stupor and Coma, CNS Series, Philadelphia:Davis (1982), which is hereby incorporated by reference. Conscious patients include those who have a capacity for reliable, reproducible, interactive behavior evidencing awareness of self or the environment. Conscious patients include patients who recover consciousness with less severe brain injury but who, because of their impaired cognitive function, do not reach independent living. Conscious patients do not include those who exhibit wakefulness but lack interaction (e.g., those deemed to be in a persistent vegetative state).

The selected subject who is conscious after exposure to a head trauma may be asymptomatic of any visible symptoms of traumatic brain injury. Conversely, the selected subject may exhibit various symptoms of brain injury and cognitive dysfunction.

This is in contrast to a subject who is unconscious at the time of the obtaining, as indicated by conditions such as a concussion or intracranial hemorrhage (e.g. intra-axial hematoma, epidural hematoma, and subdural hematoma).

As indicated above, the subject exposed to head trauma may exhibit extra-cranial injuries. Exemplary extra-cranial injuries include open head injuries, such as a visible assault to the head. Extra-cranial injuries may result from a gunshot wound, an accident or an object going through the skull into the brain (“missile injury to the brain”). This type of brain injury is likely to damage a specific area of the brain.

Alternatively, the subject exposed to a head trauma may exhibit only superficial external injuries or no extra-cranial injuries. In this instance, the subject may have no visible injury (e.g. a closed head injury), or may exhibit those symptoms by deficits in attention, intention, working memory, and/or awareness as described herein. mTBI may also include or result in any one, or more, of the following: cognition impairment; language impairment; conduct disorder; motor disorder; and any other neurological dysfunction. mTBI may occur with no loss of consciousness and possibly only a dazed feeling or confused state lasting a short time.

A brain injury may occur when there is a blow to the head as in a motor vehicle accident or a fall. In this case, the skull hits a stationary object and the brain, which is inside the skull, turns and twists on its axis (the brain stem), causing localized or widespread damage. Also, the brain, a soft mass surrounded by fluid that allows it to “float,” may rebound against the skull resulting in further damage.

In response to the head trauma, changes occur in the brain, which require monitoring to prevent further damage. The brain's size frequently increases after a severe head injury. This is called brain swelling and occurs when there is an increase in the amount of blood to the brain. Later in the illness, water may collect in the brain, which is called brain edema. Both brain swelling and brain edema result in excessive pressure in the brain called intracranial pressure (“ICP”).

Even mTBI may result in persisting debility, such as post-traumatic epilepsy, persistent vegetative state, or post-traumatic dementia in the absence of proper treatment. Other complications and late effects of brain injury include, but are not limited to, coma, meningitis, post-traumatic epilepsy, post-traumatic dementia, degeneration of nerve fibers, post-traumatic syringomyelia, or hemorrhage, for example. Although medical care administered may be minimal in the context of mTBI, persons with brain injury without coma may experience symptoms and impairments similar to those suffered by the survivor of a severe brain injury.

As used herein, the term “sample” in the context of the present invention is a body fluid sample, which can be any fluid sample containing HDL particles. Of particular interest are samples that are serum, plasma, or whole blood. Those skilled in the art will recognize that plasma or whole blood, or a sub-fraction of whole blood, may be used. While cerebrospinal fluid also can be used, in certain embodiments of the invention the term “body fluid sample” specifically excludes CSF.

The body fluid sample described may be obtained by use of a standard blood draw, as disclosed in U.S. Pat. No. 4,263,922 to White, which is hereby incorporated by reference in its entirety. Generally, in a standard blood draw, blood is drawn through a needle assembly and handle system into a collection tube. Subsequent to the blood draw, the needle assembly and the handle are removed from an end of the tube and a separate cap is fitted over each end of the tube to retain the blood sample in the tube for analysis. In the case of humans, a finger prick with a lancet or a blood draw via standard venipuncture are also convenient methods to obtain a body fluid sample.

Upon obtaining a blood sample from an individual who has suffered a head trauma, the drawn blood is preferably exposed immediately to an anticoagulant to preclude coagulation thereof. Known anticoagulants include without limitation heparin, EDTA, D-Phe-Pro-Arg chloromethyl ketone dihydrochloride (“PPACK”), and sodium citrate. Other anticoagulants may also be used.

The body fluid sample may be obtained prior to determining whether the selected subject has undergone a head trauma. This may be useful in instances where there are no witnesses to the head trauma incident that inflicted the potential mTBI to the subject.

The determination of whether the subject has suffered mTBI can be completed immediately following exposure to head trauma, or at any time thereafter. In certain embodiments, the determination of mTBI injury is completed by obtaining body fluid samples as soon as possible or immediately after exposure to head trauma, e.g., within the first hour after the injury. In other embodiments, the body fluid sample may be obtained from the subject up to 24 hours after the trauma, preferably within about six hours after the trauma occurs. Additional body fluid samples may be further obtained within hours, days, or weeks after exposure to a head trauma, i.e., as a means to monitor recovery from mTBI.

In accordance with the present invention, the biomarker used to determine whether a subject has suffered an mTBI is the presence of smaller than normal HDL particles, the presence of larger than normal HDL particles, or both. Normal serum contains a number of lipoprotein particles which are characterized according to their density, namely, chylomicrons, VLDL, LDL and HDL. They are composed of free and esterified cholesterol, triglycerides, phospho lipids, several other minor lipid components, and protein. LDL transports lipid soluble materials to the cells in the body, while HDL transports these materials to the liver for elimination. Normally, these lipoproteins are in balance, ensuring proper delivery and removal of lipid soluble materials. HDL particles function as antioxidants and as components of innate immunity. Different subclasses of HDL particles have distinct roles, with smaller HDL having a particular role as lipid acceptors and antioxidants.

Under normal conditions, a natural HDL particle is a solid with its surface covered by a phospholipid bilayer that encloses a hydrophobic core. In its nascent or newly secreted form, the particle is disk-shaped and accepts free cholesterol into its bilayer. Cholesterol is esterified by the action of lecithin cholesterol acyltransferase (“LCAT”) and is moved into the center of the disk. The movement of cholesterol ester to the center is the result of space limitations within the bilayer. The HDL particle “inflates” to a spheroidal particle as more and more cholesterol is esterified and moved to the center. Cholesterol ester and other water insoluble lipids which collect in the “inflated core” of the HDL are then cleared by the liver.

HDL particles have been defined according to several different nomenclature based on different methods for separating the particles according to their physical properties. As defined in Rosenson et al., “HDL Measures, Particle Heterogeneity, Proposed Nomenclature, and Relation to Atherosclerotic Cardiovascular Events,” Clinical Chemistry 57(3):392-410 (2011) (“Rosenson”), which is hereby incorporated by reference in its entirety, HDL particles can be classified as described in Table 1 below.

TABLE 1
Nomenclature for HDL Particles and Their Size Ranges

| Rosenson Proposal | HDL-VL | HDL-L | HDL-M | HDL-S | HDL-VS |
|---|---|---|---|---|---|
| size range (nm) | 12.9-9.7 | 9.7-8.8 | 8.8-8.2 | 8.2-7.8 | 7.8-7.2 |
| Gradient Gel | HDL2b | HDL2a | HDL3a | HDL3b | HDL3c |
| Electrophoresis | | | | | |
| size range (nm) | 12.9-9.7 | 9.7-8.8 | 8.8-8.2 | 8.2-7.8 | 7.8-7.2 |
| 2-D Gel | α-1 | α-2 | α-3 | α-4 | pre β-1 |
| Electrophoresis | | | | | |
| size range (nm) | 11.2-10.8 | 9.4-9.0 | 8.5-7.5 | 7.5-7.0 | 6.0-5.0 |
| NMR | Large | Medium HDL-P | | Small HDL-P | |
| | HDL-P | | | | |
| size range (nm) | 12.9-9.7 | 9.7-8.8 | 8.8-8.2 | 8.2-7.8 | 7.8-7.2 |

As used herein, the term “smaller than normal” HDL particles refers to HDL particles of less than 7.2 nm as measured by gradient gel electrophoresis, gel filtration chromatography, or NMR. This term also refers to HDL particles of about 5.0 nm or less as measured by 2-D gel electrophoresis (see Asztalos et al., “Distribution of ApoA-I-Containing HDL Subpopulations in Patients With Coronary Heart Disease,” Arterioscler. Thromb. Vase. Biol. 20:2670-2676 (2000), which is hereby incorporated by reference in its entirety). These smaller than normal HDL particles are poorly lipidated, typically having a lipid content of less than about 35 weight percent. All such particles, regardless of their method of separation, can be used to diagnose mTBI after exposure to head trauma.

As used herein, the term “larger than normal” HDL particles refers to HDL particles greater than 12.9 nm as measured by gradient gel electrophoresis, gel filtration chromatography, or NMR. This term also refers to HDL particles of greater than 11.2 nm as measured by 2-D gel electrophoresis (see Asztalos et al., “Distribution of ApoA-I-Containing HDL Subpopulations in Patients With Coronary Heart Disease,” Arterioscler. Thromb. Vase. Biol. 20:2670-2676 (2000), which is hereby incorporated by reference in its entirety). These larger than normal HDL particles are highly lipidated, typically having a lipid content of at least about 65 weight percent. All such particles, regardless of their method of separation, can be used to diagnose mTBI after exposure to head trauma.

As described herein, detection of smaller than normal HDL particles, larger than normal HDL particles, or both, can be carried out using a body fluid sample obtained from an individual exposed to head trauma, and then used to diagnose mTBI in that individual.

The smaller than normal or larger than normal HDL particles may be detected in a number of ways, including purification or separation of the particles based on their size, followed by immunoassay. Gel filtration chromatography and conventional preparative gradient gel electrophoresis are two of the most popular means employed for biomolecule purifications.

In gel filtration chromatography, a buffer flows through a matrix in a column. A sample of biomolecule mixture applied over the matrix is carried across the matrix by the flow of the buffer. There are numerous pores existing on the beads of the matrix. The separation of biomolecules relies on movement of the biomolecules into and out of the pores. The biomolecules at sizes larger than that of the pores cannot enter the pores and move rapidly across the matrix. The biomolecules at sizes smaller than that of the pores enter and leave the pores repeatedly and therefore remain in the matrix longer. A separation between a group of large biomolecules and a group of small ones can be achieved collecting the biomolecules into separated fractions. This separation allows for HDL particles at approximately less than 7.2 nm or greater than 12.9 nm to be distinguished from one another and from other HDL particles that fall between these sizes (see Table 1 above). In this way, the presence or absence of the smaller than 7.2 nm HDL particles, larger than 12.9 nm HDL particles, or both, can be determined. Detection of particles can be achieved using an immunoassay against proteins, such as ApoA1, present in the particle of interest.

In conventional preparative gradient gel electrophoresis, buffer does not flow through a gel matrix. However, all biomolecules have to travel across the pores of the gel matrix. Movement of biomolecules is driven by an interaction between a net charge of the biomolecules and an electric potential applied on the gel matrix. The migration rate of a given biomolecule in the gel matrix is determined by its size, its shape, its net charge, the pore size of the gel matrix, and the potential difference of the electric potential. Thus, different biomolecules in a mixture can be distinctly separated from each other at high resolution by sequentially collecting eluted fractions from a preparative gel apparatus. Detection of particles can be achieved using an immunoassay against proteins, such as ApoA1, present in the particle of interest, or via denistometry subsequent to staining with an appropriate dye or stain (e.g., silver or Coomassie Blue).

The smaller than normal HDL particles can also be detected in a 2-D gel electrophoresis, where the particles appear at about 5.0 nm or smaller (see Asztalos et al., “Distribution of ApoA-I-Containing HDL Subpopulations in Patients With Coronary Heart Disease,” Arterioscler. Thromb. Vasc. Biol. 20:2670-2676 (2000), which is hereby incorporated by reference in its entirety). Similarly, the larger than normal HDL particles can be detected in a 2-D gel electrophoresis, where the particles appear at about 11.3 nm or greater. In a 2-D gel electrophoresis, the HDL particles are first separated according to charge into pre-β, α, and pre-α mobility particles. Then plasma can be applied to agarose gels and electrophoresed in a vertical slab gel electrophoresis unit. Gel strips can be cut out, placed, and sealed (with 65° C. agarose) on the top of the nondenaturing 3% to 34% concave gradient polyacrylamide gels, followed by electrophoresis in the second dimension. Detection of particles can be achieved using an immunoassay against proteins, such as ApoA-1. One exemplary approach for the 2-D gel electrophoresis is described in Asztalos et al., “Distribution of ApoA-I-Containing HDL Subpopulations in Patients With Coronary Heart Disease,” Arterioscler Thromb Vasc Biol. 20:2670-2676 (2000), which is hereby incorporated by reference in its entirety.

In each of the above-identified procedures, detection of the HDL particles of interest can be carried out by immunoassay against any protein target that is present in or on these particles, including without limitation ApoA-1, apolipoprotein J, apolipoprotein L1, apolipoprotein F, paraoxonase 1, phospholipid transfer protein, platelet-activating factor acetylhydrolase (also termed lipoprotein-associated phospholipase A2). Other less abundant proteins in these HDL particles include apolipoprotein AII, apolipoprotein D, apoliprotein M, serum amyloids A1, A2, and A4, apolipoprotein CI, apolipoprotein CII, and apolipoprotein E.

ApoA-1 is a particularly useful protein target. ApoA-1 is a 28 kDa apolipoprotein that is primarily synthesized in the liver and small intestine, and is the major protein component of HDL. An exemplary ApoA-1 amino acid sequence is provided at Genbank accession NM-000039, which is hereby incorporated by reference in its entirety.

Detection of these protein targets for the HDL particles of interest can be achieved using polyclonal or monoclonal antibodies, as well as antibody fragments thereof. Polyclonal antibodies and fragments thereof can be raised according to known methods by administering the appropriate antigen or epitope to a host animal selected, e.g., from pigs, cows, horses, rabbits, goats, sheep, and mice, among others, and then recovering serum (containing the antibodies) from the host animal. Monoclonal antibodies can be prepared using hybridoma methods, such as those described by Kohler and Milstein, “Continuous Cultures of Fused Cells Secreting Antibody of Predefined Specificity,” Nature 256:495-7 (1975), which is hereby incorporated by reference in its entirety. Alternatively monoclonal antibodies can also be made using recombinant DNA methods as described in U.S. Pat. No. 4,816,567 to Cabilly et al., which is hereby incorporated by reference in its entirety, or phage display libraries as described by McCafferty et al., “Phage Antibodies: Filamentous Phage Displaying Antibody Variable Domains,” Nature 348:552-554 (1990), Clackson et al., “Making Antibody Fragments Using Phage Display Libraries,” Nature 352:624-628 (1991), and Marks et al., “By-passing Immunization: Human Antibodies from V-gene Libraries Displayed on Phage,” J. Mol. Biol. 222:581-597 (1991), which are hereby incorporated by reference in their entirety.

Exemplary antibody binding portions include, without limitation, Fab fragments, F(ab)₂ fragments, Fab′ fragments, F(ab′)₂ fragments, Fd fragments, Fd′ fragments, Fv fragments, minibodies, e.g., 61-residue subdomains of the antibody heavy-chain variable domain (Pessi et al., “A Designed Metal-binding Protein with a Novel Fold,” Nature 362:367-369 (1993), which is hereby incorporated by reference in its entirety), and domain antibodies (dAbs) (see, e.g., Holt et al., “Domain Antibodies: Proteins for Therapy,” Trends Biotechnol. 21:484-90 (2003), which is hereby incorporated by reference in its entirety). These antibody fragments can be made by conventional procedures, such as proteolytic fragmentation procedures, as described in J. Goding, MONOCLONAL ANTIBODIES: PRINCIPLES AND PRACTICE 98-118 (1984), which is hereby incorporated by reference in its entirety. In addition, single chain antibodies are also suitable for the present invention (e.g., U.S. Pat. No. 5,476,786 to Huston and U.S. Pat. No. 5,132,405 to Huston & Oppermann; Huston et al., “Protein Engineering of Antibody Binding Sites: Recovery of Specific Activity in an Anti-digoxin Single-chain Fv Analogue Produced in Escherichia coli,” Proc. Nat'l Acad. Sci. USA 85:5879-83 (1988); U.S. Pat. No. 4,946,778 to Ladner et al.; Bird et al., “Single-chain Antigen-binding Proteins,” Science 242:423-6 (1988); Ward et al., “Binding Activities of a Repertoire of Single Immunoglobulin Variable Domains Secreted from Escherichia coli,” Nature 341:544-6 (1989), each of which is hereby incorporated by reference in its entirety). Single chain antibodies are formed by linking the heavy and light immunoglobulin chain fragments of the Fv region via an amino acid bridge, resulting in a single chain polypeptide.

Antibody mimics can also be used including, without limitation, polypeptide scaffolds containing one or more variable regions that bind specifically to ApoA-1, or ApoA-1-binding nucleic acid aptamers.

Examples of antibodies against ApoA-I are described in, e.g., Curtiss et al., “The Conformation of Apolipoprotein A-I in High-density Lipoproteins Is Influenced by Core Lipid Composition and Particle Size: A Surface Plasmon Resonance Study,” Biochemistry 39:5712-5721 (2000); Bustos et al., “Monoclonal Antibodies to Human Apolipoproteins: Application to the Study of High Density Lipoprotein Subpopulations,” Clin. Chim. Acta 299:151-167 (2000); Marcel et al., “Lipid Peroxidation Changes the Expression of Specific Epitopes of Apolipoprotein A-I,” J. Biol. Chem. 264:19942-19950 (1989); McVicar et al., “Characteristics of Human Lipoproteins Isolated by Selected-affinity Immunosorption of Apolipoprotein A-I,” Proc. Nat'l Acad. Sci. USA 81:1356-1360 (1984); Miyazaki et al., “A New Sandwich Enzyme Immunoassay for Measurement of Plasma Pre-beta1-HDL Levels,” J. Lipid Res. 41:2083-2088 (2000); Fielding et al., “Unique Epitope of Apolipoprotein A-I Expressed in Pre-beta-1 High-density Lipoprotein and Its Role in the Catalyzed Efflux of Cellular Cholesterol,” Biochemistry 33:6981-6985 (1994), each of which is hereby incorporated by reference in its entirety. Commercially available anti-human ApoA-1 Mab 3A11-1A9 is available from Sigma-Aldrich (product WH0000335M1), and commercially available ELISA kits for detection of human ApoA-1 are available from Abnova (product H000035-AP21) and Mabtech (products 3710-1A-20 and 3710-1H-20), as well as other commercial suppliers. Any one of these products can be used to detect presence of the smaller than normal HDL particles, larger than normal HDL particles, or both.

The determination of mTBI can be made based solely on the presence of or an increased level of the smaller than normal HDL particles, solely on the presence of or an increase level of the larger than normal HDL particles, or both. In addition, the determination can also be based on additional diagnostic markers or biomarkers. There are a number of additional biomarkers that can be used to assist in detecting or diagnosing mTBI following a head trauma. Biomarker S100B is a brain protein that can be used to predict the necessity of obtaining a head CT in a concussion patient. S100B is defined as a protein from the group consisting of the so-called “S100” proteins which, as their name implies, have the property of remaining in solution even at 100% saturation with ammonium sulphate at neutral pH (solubility 100%). They belong to the calcium-binding proteins, which are usually localized in cytoplasma. However, some S100 proteins, including S100B, also occur in the extracellular space. S100 proteins and their known properties, functions, and positive or negative effects in various pathological processes have been thoroughly studied, with particular emphasis those of the brain and central nervous system (Donato, “S100: A Multigenic Family of Calcium-Modulated Proteins of the EF-Hand Type With Intracellular and Extracellular Functional Roles,” Int. J. Biochem. Cell Biol. 33:637-668 (2001); Donato, “Functional Roles of S100 Proteins, Calcium-Binding Proteins of the EF-Hand Type,” Biochim. Biophys. Acta. 1450:191-231 (1999), which are hereby incorporated by reference in their entireties). Detecting elevated S100B can be carried out according to the procedures described in Biberthaler et al., “Serum S-100B Concentration Provides Additional Information for the Indication of Computed Tomography in Patients After Minor Head Injury: A Prospective Multicenter Study,” Shock 25:446-453 (2006), which is hereby incorporated by reference in its entirety.

A subject who is conscious after exposure to a head trauma may be asymptomatic of any visible diagnostic markers of traumatic brain injury. Conversely, the subject may exhibit various diagnostic markers of brain injury and cognitive dysfunction.

Diagnostic markers that can be used to determine whether the subject that was exposed to head trauma has mTBI may include one or more than one of the following: memory loss; pupil dilation; convulsions; distorted facial features; fluid draining from nose, mouth, or ears; fracture in the skull or face; bruising of the face; swelling at the site of injury; scalp wound; impaired hearing, smell, taste, or vision; inability to move one or more limbs; irritability; personality changes; unusual behavior; confusion; drowsiness; low breathing rate; drop in blood pressure; restlessness, clumsiness; lack of coordination, severe headache, slurred speech; stiff neck; and vomiting. A mild brain injury that occurs without loss of consciousness may leave a subject with merely a dazed feeling or confused state lasting a short time.

Brain injury symptoms frequently manifest themselves in combined deficits of attention, intention, working memory, and/or awareness. As used herein, attention refers to the cognitive function that provides the capacities for selection of internal or external stimuli and thoughts, supports the preparation of intended behaviors (e.g., speeds perceptual judgments and reaction times), and supports the maintenance of sustained cognition or motor behaviors (e.g., the focusing of attention). Intention, as used herein, refers to the mechanism of response failures (i.e., lack of behavioral interaction) which is not due to a perceptual loss (i.e., intention is the cognitive drive linking sensory-motor integration to behavior). Intention deficits include failure to move a body part despite intact motor pathways, awareness, and sensory processing as demonstrated by neurophysio logical and neuropsycho logical evaluation. Another example of a patient's intention deficit is a failure to initiate action of any kind despite evidence of awareness or action produced by stimulation. Loss of intention is a disorder of cognitive function, as defined herein, and is a major division of the neuropsycho logical disorder of neglect, which may be present in many patients with cognitive loss following brain injury caused by a head trauma. Working memory, as used herein, refers to the fast memory process required for on-line storage and retrieval of information, including processes of holding incoming information in short-term memory before it can be converted into long-term memory and processes which support the retrieval of established long-term (episodic) memories. Deficits in awareness relate to impaired perceptual awareness, as described above. Clinical signs of these brain injuries also include profound hemi-spatial neglect, disorders of motor intention, disorders of impaired awareness of behavioral control, or apathy and cognitive slowing.

After a subject who was exposed to head trauma is subjected to the methods of the present invention to evaluate the presence of mTBI, the result of such evaluation will determine the course of treatment, if any, for the tested subject.

If the result of the method for mTBI detection is negative (i.e., few, if any, smaller than normal HDL particles or larger than normal HDL particles are detected), then the subject does not have mTBI and, therefore, can resume normal daily activities fairly soon.

If, on the other hand, the result of the method for mTBI detection is positive (i.e., smaller than normal HDL particles are detected, larger than normal HDL particles are detected, or both), then the subject should be treated for mTBI, including rest and refraining from all potentially dangerous activities that could inflict additional head trauma. HDL particle measurements can be re-evaluated according to the method of the present invention at various time periods after the head trauma. For example, subsequent evaluations can occur approximately 24 hours, 48 hours, 72 hours, 96 hours, 120 hours, 144 hours, and/or one week after the trauma is inflicted, and anytime thereafter. This repeated course of testing, along with evaluation of diagnostic indicia of mTBI, will help evaluate the proper treatment, and whether the subject is ready to resume normal activities. The determination may be used as a method to monitor follow up treatment, by testing the selected subject's HDL particle levels at various time points during and after treatment for a previous head trauma.

If the result of the method described herein indicates a borderline positive result (i.e., only a small fraction of smaller than normal HDL particles, larger than normal HDL particles, or both, are detected), then other diagnostic markers described herein can be used to assess the subject's potential need for treatment.

Exemplary methods of treatment include withholding physically strenuous activity or all activity for one week, or until smaller than normal HDL particle levels, larger than normal HDL particles, or both, retreat below a particular threshold value.

The term “treating” or “treatment” as used herein, should be understood as partially or totally preventing, inhibiting, attenuating, ameliorating or reversing one or more symptoms or cause(s) of mTBI injury, as well as symptoms, diseases or complications accompanying mTBI injury.

A further aspect of the invention includes a kit that can be used to detect both S100B levels and smaller and/or larger than normal HDL particles in a single body fluid sample. The kit may include one or more binding partner reagents that bind specifically to S100B, ApoA-1, and reagents for separation of HDL particles using, e.g., gradient gel electrophoresis, gel filtration chromatography, or 2-D gel electrophoresis. The kit may further include one or more of the following: a solid surface, reagents for detecting a label, and instructions for carrying out detection of S100B, as well as guidelines for identifying the existence of mTBI based on the results of using the kit on a body fluid sample and identifying whether a CT scan is warranted based on the results of using the kit on the body fluid sample.

### EXAMPLES

The following examples are provided to illustrate embodiments of the present invention but are by no means intended to limit its scope.

### MATERIALS AND METHODS FOR EXAMPLES

Subject Enrollment: All studies received institutional review board approval. Informed consent was obtained prior to enrolling all subjects. All mTBI patients enrolled met a consensus definition of mTBI (American Congress of Rehabilitation Medicine Mild Traumatic Brain Injury Committee of the Head Injury Interdisciplinary Special Interest Group, “Definition of Mild Traumatic Brain Injury,” J. Head Trauma Rehabilitation 8:86-87 (1993), which is hereby incorporated by reference in its entirety).

Subjects with mTBI were selected from a large parent cohort accrued through the University of Rochester Medical Center Emergency Department (URMC ED). A total of 1910 patients were enrolled and a subset of 690 consented to having blood drawn. Three months after the initial URMC ED visit, post-concussive scores were determined by telephone interview using the Rivermead Post Concussion Questionnaire (RPCQ) (King et al., “The Rivermead Post Concussion Symptoms Questionnaire: A Measure of Symptoms Commonly Experienced after Head Injury and its Reliability,” J. Neurol. 242:587-592 (1995), which is hereby incorporated by reference in its entirety). Post-concussive symptom scores range from 0 (asymptomatic) to 64 (very symptomatic). For proteomic studies 3 symptomatic (mean PCS score=25, SD 10) and 3 asymptomatic (PCS score=0) subjects were selected from the cohort. For the measurement of apoA-1 concentration, 100 different mTBI subjects were selected from the parent cohort. Subjects with extra cranial trauma only were enrolled from patients presenting to the URMC ED who had an isolated extremity injury requiring an x-ray. Subjects were excluded if they had a blow to the head as part of their injury mechanism, or any symptoms of TBI. Sera from uninjured patients were obtained from healthy volunteers without current, acute health problems.

For the S100B studies, subjects were enrolled as part of a multicenter study of patients presenting to the emergency department with clinically defined mTBI. Participating institutions included URMC, Erie County Medical Center, SUNY Upstate Medical University, Albany Medical Center, Guthrie Health and Medical Center, and Bassett Hospital. Uninjured subjects for S100B studies were enrolled from volunteers presenting to the clinical lab at URMC for routine outpatient blood draws.

Serum Collection and Handling: For mTBI subjects, whole blood was collected by venipuncture into serum separator tubes and placed on ice. Samples were centrifuged to separate serum which was first frozen at −20° C. and then transferred to a −80° C. freezer. For all other subjects, blood was collected and processed similarly but was frozen at −80° C.

Proteomics: Serum samples were thawed on ice, depleted of high abundance proteins using the ProteoExtract Albumin/IgG Removal Kit (Calbiochem, Gibbstown N.J.), and then concentrated with Vivaspin 500Max columns (Sartorius, Edgewood N.Y.). Proteins were separated by two-dimensional gel electrophoresis with isolectric focusing and SDS-PAGE for the first and second dimensions respectively. Proteins were stained and staining intensity of individual spots was measured and analyzed using PDQuest software (Bio-Rad, version 7.4.0). Spots identified as different between groups were cut from gels and analyzed by the Proteomics and Mass Spectrometry Core Facility at Cornell University for identification by MALDI-TOF/TOF mass spectroscopy.

Quantification of Proteins: S100B concentrations were measured with an electrochemiluminescence immunoassay kit (Elecsys S100; Roche Diagnostics, Mannheim, Germany).

Fast Protein Liquid Chromatography: Lipoproteins present in pooled sera from control and from TBI patients were separated by fast protein liquid chromatography (FPLC) gel filtration using a Superose 6 HR 10/30 column (Pharmacia Biotech, Inc.) and a flow rate of 0.4 ml/min. Cholesterol and triglyceride concentrations were measured in each fraction using the Cholesterol E kit (Wako Chemicals) and Infinity Triglyceride kit (Thermo Scientific), respectively.

Immunoblotting: Serum was separated by polyacrylamide gel electropheresis (“PAGE”) followed by electrophoretic transfer to PVDF membranes. Membranes were blocked, incubated overnight with anti apoA-1 antibody, washed and incubated with appropriate horseradish peroxidase conjugated secondary antibody. Antibody binding was detected by chemiluminescence using Western Lightning ECL Plus kit (Perkin Elmer, MA).

For FPLC fractions, SDS-PAGE was used for protein separations. The primary antibody was mouse monoclonal anti apoA-1 (Abcam, MA). Band density was quantified with Image J software (National Institute of Health, MD). Human HDL (100 ng protein) isolated by ultracentrifugation was coelectrophoresed as a positive control on each gel and assigned a value of 100% absorbance. The absorbance of each band was expressed relative to the HDL control. Corresponding fractions from both mTBI and uninjured subjects were evaluated on each gel to facilitate comparisons.

For gradient gel electrophoresis, 4-20% (w/v) polyacrylamide gradient gels (BioRad, CA) were employed under non-denaturing conditions. The primary antibody was goat polyclonal anti-apoA-1 (ThermoScientific). Sample volumes were adjusted so that the same total amount of apoA-1 was loaded in each lane. HMW-Native molecular weight marker (GE Healthcare Biosciences, Pittsburgh, Pa.) was used to estimate particle size.

Statistics: Two-tailed Student's t-tests were used to determine differences between groups for S100B serum concentrations. Receiver operator characteristic curve analysis was performed using GraphPad Prism 5 for Windows (La Jolla, Calif.). For all tests, a value of p<0.05 was considered significant.

### Example 1

### HDL Particle Concentrations as a Marker for mTBI

HDL particles are continually remodeled and play an important role in reverse cholesterol transport (RCT) (Lewis et al., “New Insights into the Regulation of HDL Metabolism and Reverse Cholesterol Transport,” Circ. Res. 96:1221-1232 (2005), which is hereby incorporated by reference in its entirety). The biological behavior of individual HDL particles is determined mainly by the physiochemical structure of the particle and its associated lipids and proteins. HDL particles also function as antioxidants and as components of innate immunity (Barter et al., “Anti-inflammatory Properties of HDL,” Circ. Res. 95:764-772 (2004); Kontush et al., “Functionally Defective High-Density Lipoprotein: A New Therapeutic Target at the Crossroads of Dyslipidemia, Inflammation, and Atherosclerosis,” Pharmacol. Rev. 58:342-374 (2006), which are hereby incorporated by reference in their entireties). Different subclasses of HDL particles have distinct roles with smaller, lipid-poor HDL having a particular role as lipid acceptors and antioxidants (Davidson et al., “Proteomic Analysis of Defined HDL Subpopulations Reveals Particle-Specific Protein Clusters: Relevance to Antioxidative Function,” Arterioscler. Thromb. Vasc. Biol. 29:870-876 (2009), which is hereby incorporated by reference in its entirety).

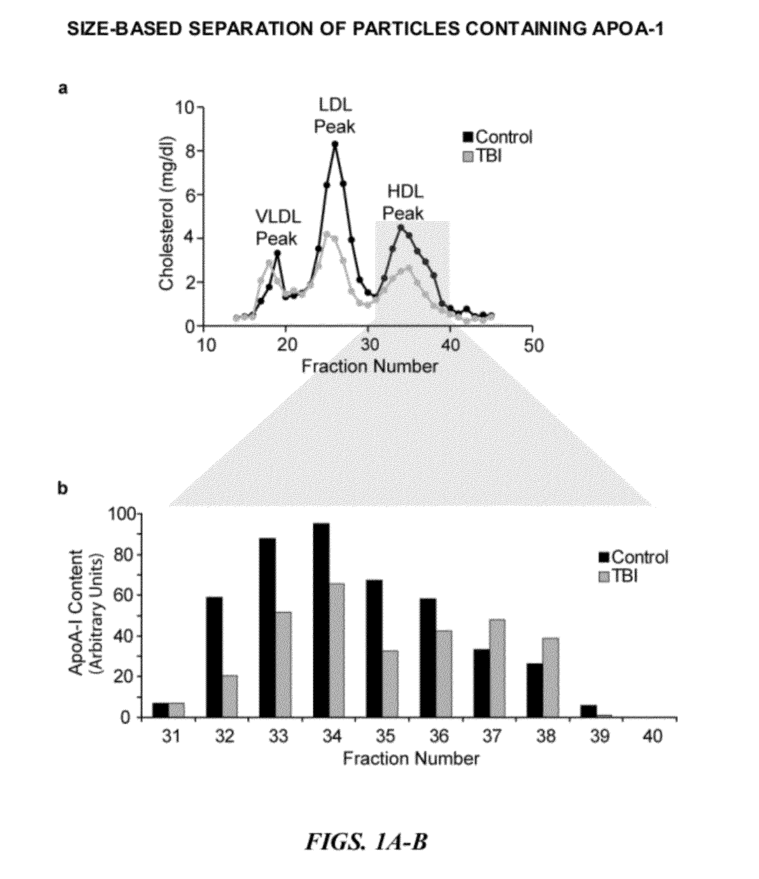

Gel filtration chromatography (FPLC) of serum was used to separate particles based on size, as shown in FIG. 1. Total cholesterol was higher in control sera than in the mTBI sera mainly due to differences in LDL cholesterol. Differences in HDL cholesterol curves between the two groups were observed, identifying a broader size distribution of HDL in the TBI group compared with control. Two peaks were observed in sera from mTBI: one matching the main peak seen in control sera, and a second, smaller peak, centered at fraction 37. As this method separates lipoproteins based on size, these results demonstrate the presence of a unique population of smaller than normal HDL particles in sera from mTBI.

HDL separated by gradient gel electrophoresis is typically divided into 5 subclasses ranging in size from 7.2 to 12.9 nm (Rosenson et al., “HDL Measures, Particle Heterogeneity, Proposed Nomenclature, and Relation to Atherosclerotic Cardiovascular Events,” Clinical Chemistry 57(3):392-410 (2011); Barter et al., “High Density Lipoproteins (HDLs) and Atherosclerosis: The Unanswered Questions,” Atherosclerosis 168:195-211 (2003), which is hereby incorporated by reference in its entirety). While HDL particles as small as 7.1 nm have been studied in in vitro systems, they are not typically observed in human serum (Barter et al., “High Density Lipoproteins (HDLs) and Atherosclerosis: The Unanswered Questions,” Atherosclerosis 168:195-211 (2003), which is hereby incorporated by reference in its entirety). Serum derived from mTBI patients differed from that of uninjured subjects by having more distinctive bands, including bands running at 7.2 nm and smaller and bands running between about 13 nm and 17.0 nm. These results indicate that mTBI may stimulate the synthesis and secretion of nascent HDL and the scavenger role of HDL

The identification of small, poorly lipidated HDL particles and larger, lipid-rich HDL particles following mTBI are novel findings. These data have important implications for TBI biomarker development. Most prior TBI biomarker studies have sought to detect central nervous system (CNS) specific proteins that have leaked into blood as a consequence of trauma to brain. This approach has largely failed perhaps due to the effects of the BBB. The BBB is an anatomic structure which prevents the passive movement of large molecules (>500 Daltons) into and out of the CNS. Because BBB function is often but not always impaired after TBI (Morganti-Kossmann et al., “TGF-beta is Elevated in the CSF of Patients with Severe Traumatic Brain Injuries and Parallels Blood-Brain Barrier Function,” J. Neurotrauma 16:617-628 (1999); Blyth et al., “Validation of Serum Markers for Blood-Brain Barrier Disruption in Traumatic Brain Injury,” J. Neurotrauma. 26:1497-1507 (2009), which are hereby incorporated by reference in their entireties), its functional status has likely confounded previous efforts to consistently identify CNS proteins in blood after brain injury. This may have led to poor diagnostic accuracy of S100B for the clinical diagnosis of mTBI.

The alteration of peripheral lipid transport as a result of trauma to the brain is a compelling explanation that the significant observations of the present invention are part of a physiologic response to TBI rather than simply a consequence of structural brain injury. Under normal conditions, the brain produces its own cholesterol, non-essential fatty acids, and lipoproteins (Bjorkhem et al., “Brain Cholesterol: Long Secret Life Behind a Barrier,” Arterioscler. Thromb. Vasc. Biol. 24:806-815 (2004); Edmond, “Essential Polyunsaturated Fatty Acids and the Barrier to the Brain: The Components of a Model for Transport,” J. Mol. Neurosci. 16:181-193; discussion 215-121 (2001), which are hereby incorporated by reference in their entireties). TBI results in lipid damage from mechanical injury to axons and subsequent oxidative stress (Lewen et al., “Free Radical Pathways in CNS Injury,” J. Neurotrauma 17:871-890 (2000), which is hereby incorporated by reference in its entirety). It seems plausible that central lipid metabolic requirements would increase after TBI requiring greater involvement of peripheral lipid transport systems.

Interactions between peripheral and central lipid transport likely occur within the BBB itself. Peripheral HDL particles interact with the BBB via specific lipoprotein receptors located on the luminal surface of brain capillary endothelial cells but do not cross into the brain parenchyma itself (Edmond, “Essential Polyunsaturated Fatty Acids and the Barrier to the Brain: The Components of a Model for Transport,” J. Mol. Neurosci. 16:181-193; discussion 215-121 (2001), which is hereby incorporated by reference in its entirety).

### Example 2

### S100B Concentrations in mTBI Subjects

The only blood biomarker in clinical use with TBI is S100B, which is used widely in Europe but is not yet approved in the United States. Serum concentrations of this predominantly glial protein measured within 6 hours of injury are very sensitive for the identification of mTBI patients with traumatic injury detectable by cranial computed tomography scans (Biberthaler et al., “Serum 5-100B Concentration Provides Additional Information for the Indication of Computed Tomography in Patients After Minor Head Injury: A Prospective Multicenter Study,” Shock 25:446-453 (2006), which is hereby incorporated by reference in its entirety). The accuracy of this test as a surrogate for clinical diagnosis is unclear, however.

Therefore, it is proposed to use the assessment of HDL particle size distribution, particularly the presence of HDL particles smaller than about 7.2 nm or larger than about 12.9 nm as measured using gradient gel electrophoresis, gel filtration chromatography, or NMR (or 5.0 nm or 11.2 nm, respectively, as measured using 2-D gel electrophoresis), in combination with S100B serum concentrations as a measure of mTBI. It is expected that the combination of detecting the smaller and/or larger than normal HDL particles together with elevated S100B levels in serum of subjects will improve the diagnosis of mTBI, generally, as well as a subset of mTBI patients who will benefit from cranial CT scans to detect traumatic injury. This is particularly useful for individuals lacking or having only minimal external signs of trauma.

Although the invention has been described in detail for the purposes of illustration, it is understood that such detail is solely for that purpose, and variations can be made therein by those skilled in the art without departing from the spirit and scope of the invention which is defined by the following claims.

What is claimed:

1. A method of determining whether a subject has suffered a mild traumatic brain injury, said method comprising:
selecting a subject exposed to a head trauma; and
determining whether a body fluid sample obtained from the selected subject comprises smaller than normal high density lipoprotein (HDL) particles, larger than normal HDL particles, or both;
wherein detection of the smaller than normal HDL particles, larger than normal HDL particles, or both, indicates that the subject has suffered a mild traumatic brain injury.

2. The method of claim 1, wherein the body fluid sample is selected from the group consisting of serum, plasma, and whole blood.

3. The method of claim 1, wherein the body fluid sample is not cerebral spinal fluid.

4. The method of claim 1, wherein the smaller than normal HDL particles are less than about 7.2 nm as measured by gradient gel electrophoresis, gel filtration chromatography, or NMR.

5. The method of claim 1, wherein the smaller than normal HDL particles are less than about 5.0 nm as measured by 2-D gel electrophoresis.

6. The method of claim 1, wherein the larger than normal HDL particles are greater than about 12.9 nm as measured by gradient gel electrophoresis, gel filtration chromatography, or NMR.

7. The method of claim 1, wherein the larger than normal HDL particles are greater than about 11.2 nm as measured by 2-D gel electrophoresis.

8. The method of claim 1, wherein both smaller than normal HDL particles and larger than normal HDL particles are detected.

9. The method of claim 1, further comprising:
obtaining the body fluid sample from the selected subject within 24 hours of the head trauma.

10. The method of claim 9, wherein said obtaining is carried out with about six hours of the head trauma.

11. The method of claim 9, wherein said obtaining is carried out prior to said determining.

12. The method of claim 9, wherein the subject is conscious at the time of said obtaining

13. The method of claim 1, wherein the subject has extra-cranial injuries.

14. The method of claim 1, wherein the subject has no extra-cranial injuries.

15. The method of claim 1, wherein the method is carried out repeatedly in spaced intervals over a period of time.

16. The method of claim 1, wherein the method is used with an additional biomarkers other than HDL particle size to identify mTBI.

17. The method of claim 16, wherein the additional biomarker used is S100B.

18. The method of claim 1, wherein the method is used with other diagnostic markers to identify mTBI.

19. The method of claim 18, wherein the other diagnostic markers comprise one or more than one of the following: memory loss; pupil dilation; convulsions; distorted facial features; fluid draining from nose, mouth, or ears; fracture in the skull or face; bruising of the face; swelling at the site of injury; scalp wound; impaired hearing, smell, taste, or vision; inability to move one or more limbs; irritability; personality changes; unusual behavior; loss of consciousness; confusion; drowsiness; low breathing rate; drop in blood pressure; restlessness, clumsiness; lack of coordination, severe headache, slurred speech; stiff neck; and vomiting.

20. The method of claim 1, wherein the smaller than normal HDL particles contain less than about 35 wt % lipid content.

21. The method of claim 1, wherein the larger than normal HDL particles contain at least about 65 wt % lipid content.

22. The method of claim 1, wherein said determining is carried out by gradient gel electrophoresis, gel filtration chromatography, NMR, or 2-D gel electrophoresis.

23. The method of claim 1, wherein the HDL particles are detected by a protein immunoassay.

24. The method of claim 1, wherein the method is used for treating the subject for mTBI based on the levels of smaller than normal HDL particles, larger than normal HDL particles, or both, in the body fluid sample.

25. The method of claim 1, wherein treatment is withheld when the body fluid sample obtained from the subject is deficient in smaller than normal HDL particles, larger than normal HDL particles, or both.

26. The method of claim 1, wherein treatment is administered when the body fluid sample obtained from the subject contains smaller than normal HDL particles, larger than normal HDL particles, or both.
